(19) United States
(12) Patent Application Publication
(10) Pub. No.: US 2006/0089601 A1
(43) Pub. Date: Apr. 27, 2006

(54) Activation-based injection device

(76) Inventor: Francesca Dionigi, Folingo (IT)

Correspondence Address: Frank J Catalano;Gable & Gotwals, 100 W. 5th Street, 10th Floor, Tulsa, OK, 74103, US

(21) Appl. No.: 10/521,537

(22) PCT Filed: Jul. 14, 2003
(86) PCT No.: PCT/US03/21799

§ 371 Date: Jan. 14, 2005

(30) Foreign Application Priority Data
Jul. 15, 2002 (IT) RM02A00378

(51) Int. Cl. A61M 5/00

(52) U.S. Cl. 604/181

(57) ABSTRACT

An activation-based injection device includes an activation unit (1, 2, 3), a vial unit (5) containing a drug and a needle (2) with an inner point (26) which never comes in contact with the external environment. The device is activated by a simple axial shift of the needle-shield (1) against the activation head (3). The latter is transparent and contains a window (45) of lower thickness. This allows the user to check the exact position of the needle hub (21) as a proof of tamper evidence, allowing the user to immediately determine whether the device has been previously activated or tampered with and if there is a risk that the drug has been contaminated.

### BACKGROUND OF THE INVENTION

This invention relates to devices for injecting medication into a patient and more particularly concerns a device which, when combined with a vial, forms a pre-filled syringe.

Pre-filled syringes offer many advantages as they reduce both the preparation time for injections and the risk of contamination. U.S. Pat. No. 4,055,871 teaches a thermoplastic compressible reservoir which cannot be used to administer different doses of drugs. U.S. Pat. No. 3,994,296 describes a syringe in which the connection between the reservoir and the needle is accomplished by screwing the reservoir to the syringe body, causing the inner point of the needle to penetrate into the reservoir which is sealed by a thin rubber membrane. When the device is activated, the inner point of the needle remains out and is exposed to accidental contamination. In U.S. Pat. Nos. 3,841,329, 3,994,296 and 5,478,324, the device includes a central valve which, when suitably pressed, allows the drug to flow through the valve and out of the cannula. The valve makes the container much less airtight and makes storage of the drug in the vial and separated from the injector unit very critical.

In U.S. Pat. Nos. 3,994,296 and 396,589, the devices are equipped with an intact plunger that, upon injection, is pierced by a double-pointed cannula. But, the vial containing the drug and the injector unit must be assembled just before use. When the cap of the injector unit is removed, the inner point of the cannula is exposed and could accidentally prick the user.

It is, therefore, an object of this invention to provide an activation-based injection device in which the inner point of the needle never comes in contact with the external environment. Another object of this invention is to provide an activation-based injection device by which unintentional activation of the system is immediately observable. A further object of this invention is to provide an activation-based injection device which cannot be activated by accidental pressure. Yet another object of this invention is to provide an activation-based injection device which facilitates administering varying amounts or dosages of drugs.

### SUMMARY OF THE INVENTION

In accordance with the invention, an activation-based injection device has an activation head that is pre-assembled to a vial containing the drug or that can be connected to the vial by the user before the injection. The inner needle of the device remains isolated form the external environment at all times. An activation indicator enables the user to see if the device has already been activated or tampered with. The vial is preferably made of glass or non-deformable inert materials and is configured to be filled by duly adjusted production lines for both conventional pre-filled syringes and vials. In one particular configuration, this device is not reusable so as to reduce the risk of transmission of infections from one individual to another.

The device has an activation head closed at one end by a pierceable membrane. A needle-shield open at its base fits into and slides in a rail of the activation head. A rigid vial is sealed by a sliding plunger. The plunger is adapted for attachment to the closed end of the activation head for making direct contact between a pierceable membrane of the plunger and the pierceable membrane of the activation head. A needle with a double-pointed cannula is secured to a hub disposed inside the rial of the activation head. A needle-shield is slidable inside the activation head from an initial resting position to a final activation position to act on the hub and sequentially pierce the pierceable membranes with the inner point of the needle, thus connecting the cannula to the vial. An activation indicator on the rail of the activation head indicates the initial resting position of the hub of the cannula in the activation head when the pierceable membrane is intact.

### BRIEF DESCRIPTION OF THE DRAWINGS

Other objects and advantages of the invention will become apparent upon reading the following detailed description and upon reference to the drawings in which:

FIG. 1 is a perspective view of the assembled syringe ready for activation;

FIG. 2 is a perspective drawing of the syringe assembly of FIG. 1;

FIG. 3 is a cross-sectional view of the syringe of FIG. 1 ready for activation;

FIG. 4 is a cross-sectional view of the syringe of FIG. 3 activated and ready for injection after removal of the needle-shield;

FIG. 5 is a top plan view of the syringe of FIG. 1;

FIG. 6 is an elevation view of a stopped vial separate from the device which can be assembled to the activation head of the device to compose the complete device of FIG. 1;

FIG. 7 is an elevation view of the injection unit-shown in FIG. 1 separate from the vial;

FIG. 8 is a partial cross-sectional view of the non-reusable embodiment of the syringe before activation;

FIG. 9 is a partial cross-sectional view of the syringe of FIG. 8 during injection; and

FIG. 10 is a partial cross-sectional view of the syringe of FIG. 9 as it draws in air (liquid cannot be withdrawn by this version of the device).

While the invention will be described in connection with a preferred embodiment, it will be understood that it is not intended to limit the invention to that embodiment. On the contrary, it is intended to cover all alternatives, modifications and equivalents as may be included within the spirit and scope of the invention as defined by the appended claims.

### DETAILED DESCRIPTION OF PREFERRED CONFIGURATIONS

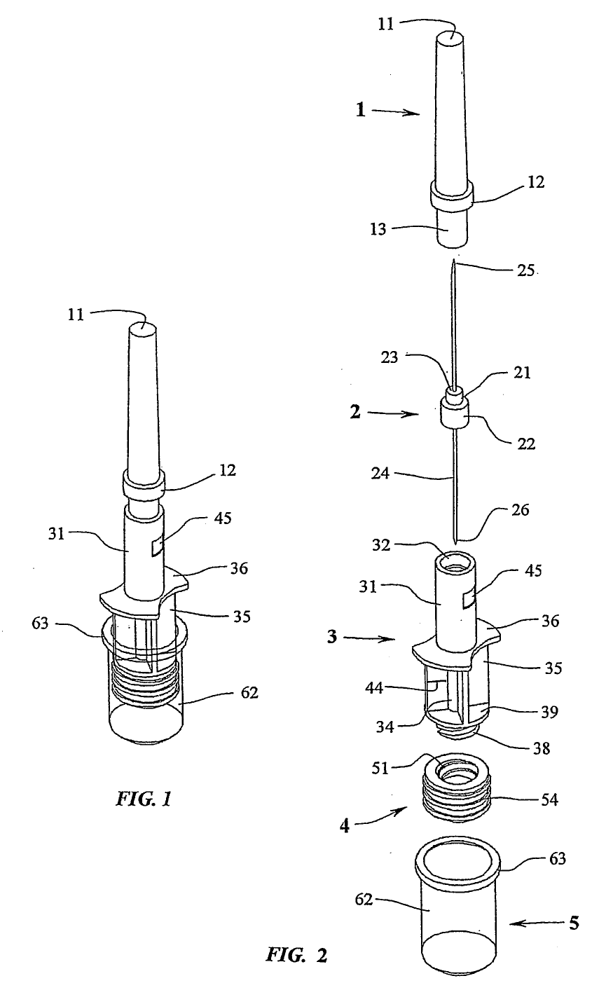

The first preferred configuration for this invention is shown in FIGS. 1 and 2. An activation head 3 made of plastic such as polypropylene accommodates a needle 2 and serves as a syringe handgrip and pressure rod as well. The needle 2, a double-pointed cannula 24 having end points 25 and 26, is secured to a plastic hub 21. A needle-shield 1 fits into the rail 31 of the activation head 3, closing its open end. The activation head 3, needle 2 and plastic hub 21 compose, all together, the injection unit 7 illustrated in FIG. 7. The injection unit 7 is secured to the drug container which is a tubular vial 5 sealed by a rubber plunger 4 which moves along the centerline of the plunger 4. The rubber on the plunger 4 is thinner in the middle so as to serve as a pierceable membrane 52.

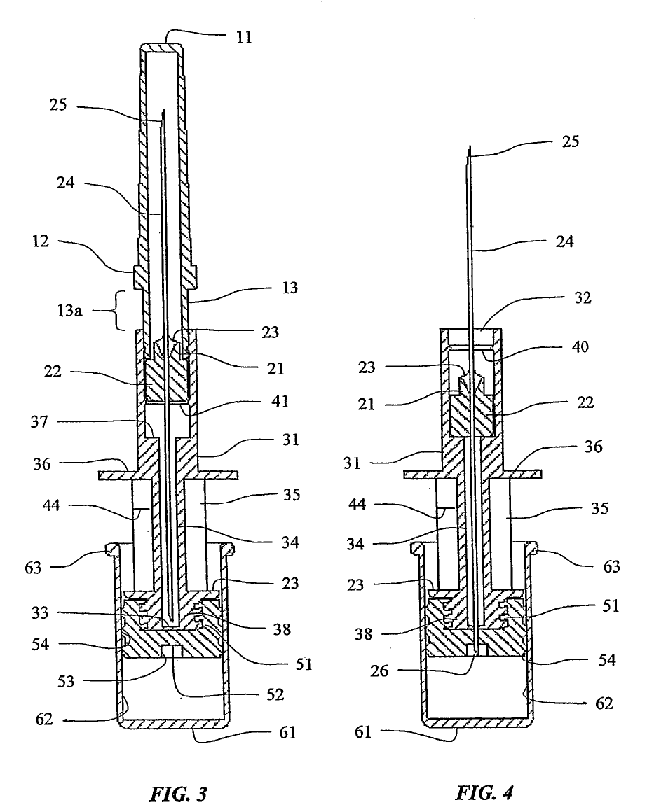

Looking at FIGS. 2 and 3, the activation head 3 has a tubular rail 31 open at one end 32 and closed at the other end by a thin membrane 33. In the preferred configuration, the closed end has a male threading 38 near the membrane 33. The rail 31 is fitted, in its inner wall, with a system that engages the hub 21 of the needle 2. In the preferred configuration, as best seen in FIGS. 3 and 4, this system is composed of two thin rings 40 and 41 placed before and after the hub 21. One ring 40 prevents the needle 2 from getting out of the rail 31, while the other ring 41 locks it so that the syringe cannot be activated unless the hub 21 is pressed hard enough to overcome the resistance offered by the ring 41. The ring 40 further locks the base 13 of the needle-shield 1 and prevents it from coming off the rail 31 before activation. The ring 41 further locks the hub 21 of the needle 2 after activation. Beyond the ring 41, the inner diameter of the rail 31 of the activation head 3 narrows to form a stop 37, which lets the cannula 24 pass through but stops the hub 21. A channel 34, extending from the stop 37 to the membrane 33, houses the non-visible part of the cannula 24 and keeps its centerline aligned during activation. In the preferred configuration, three or more radial flaps 35, best seen in FIGS. 1 and 2, stiffen the base of the activation head 3 and make it stable as it slides inside the vial 5 during injection. These flaps 35 are joined lengthways to the outer surface of the channel 34 and at both ends to the flange 36 and platform 23.

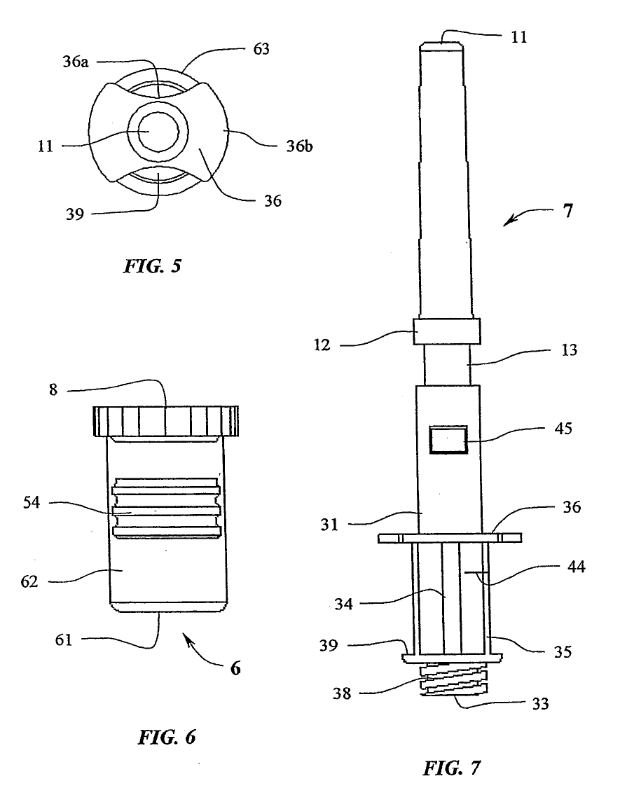

During injection, the user presses the flange 36 against the vial 5. Its shape may be customized for ergonomics or design requirements. Looking at FIG. 5, in the preferred configuration, the flange 36 is elongated in shape, with two side flaps 36b and a bottleneck 36a in the middle. The bottleneck 36a in the middle affords an easier grip on the device during activation. The flaps 36b act as rests during injection.

Returning to FIG. 2, the needle 2 is a double-pointed steel cannula 24 secured through a hub 21 made of plastic such as polypropylene or polystyrene by a sealant 23 such as epoxy glue. As seen in FIGS. 3 and 4, the inner point 26 of the cannula pierces the membranes 33 and 52 during activation and draws the drug from the reservoir 62 of the vial 5. The outer point 25 of the cannula injects the drug into the patient.

In the preferred configuration, the hub 21 has an elongation 22 so as to fit better between the rings 40 and 41 before activation and between the ring 41 and the stop 37 after activation. The plastic body of the hub 21 is available in different colors indicative of different sizes of cannulas. In this way, the user may immediately tell the characteristics of the needle fitted on the syringe from the color of the hub 21 as seen through the rail 31 of the activation head 3 window 45 which is provided along the rail 31. In this window, the wall of the rail 31 is thinner, so the user can more easily see both the position and color of the hub 21 inside. Basically, the window 45, which appears colored because of the hub 21 standing behind, indicates that the device has not been damaged or tampered with.

The needle-shield 1 shown in FIGS. 1, 2, 3 and 7, is made of plastic such as polypropylene and may be cylindrical or conical in shape. It is closed at one end 11 and open at the base 13, and completes the injection unit. The base 13 fits in the rail 31 of the activation head 3. Just above the base 13, the needle-shield 1 is fitted with a thicker ring 12, which acts as a stop during activation and as a handgrip for the user. In the preferred configuration, the needle-shield 1 is transparent, so the position and state of the needle 2 in the finished product can be checked. In addition, unlike other needle-shields of ordinary pre-filled syringes, its rigid structure protects the needle from accidental collisions or stresses that could damage it.

The vial 5 seen in FIGS. 1-6 is the reservoir for the drug to be dispensed. It is a simple pipe 62 made of glass or other materials and closed at one end 61. Its open end may be fitted with a thicker ring 63, if required by the filling machine.

The rubber plunger 4, made for instance of butyl rubber, has the membrane 52 in the middle and is provided with at least two sealing rings 54 which hermetically seal the vial 5. The plunger 4 slides inside the vial 5 in the coaxial direction. The membrane 52 splits the fastening device that joins the plunger 4 to the injection unit. In its preferred configuration, the fastening device consists of a female threading 51 matched to the male threading 38 of the activation head 3 and a small cylindrical cavity 53, the purpose of which will be specified later.

In its initial resting position shown in FIGS. 1 and 3, the membrane 33 of the activation head 3 and the membrane 52 of the plunger 4 are intact and adjacent to each other, and the cannula 24 does not make contact with the liquid contained in the vial 5. In this condition, the base 13 of the needle-shield 1 extends partly into the rail 31 of the activation head 3. The gap 13a and the position of the colored hub 21 behind the window 45 show that the syringe has not been tampered with, so the system is “tamper-evident”.

To activate the system, the needle-shield 1 must be pressed against the activation head 3. In this way, the base 13 of the needle-shield 1 presses against the hub 21 of the needle 2. The needle 2 slides in the rail 31 of the activation head 3, and the hub 21, once the resistance offered by the ring 41 is overcome, slides and stops against the stop 37. At the same time, the inner point 26 of the cannula 24 moves, piercing first the membrane 33 of the activation head 3 and then the membrane 52 of the plunger and finally stopping in the cavity 53, as is seen in FIG. 4. When the needle-shield 1 has been removed and the flange 36 of the activation head 3 is pressed against the back 62 of the vial, the liquid is forced through the cannula 24 and out of the outer point 25.

The part of the activation head 3 between the flange 36 and the platform 39 can be equipped with one or more level gauges such as a line 44 or a screen-printed graduation, a raised arrow, etc., so that the device can be used to administer both full doses and fractions of doses, such as, for instance, a pediatric dose. To do this, the user, as instructed in the drug leaflet, must activate the device, remove the needle-shield 1 and throw away part of the drug until the level gauge 44 on the activation head 3 is at the same level as the ring 63 of the vial 5. At that point, the user can administer the remaining fraction of the dose to the patient.

In a variation of the above described version of the device, the stopped vial 6 shown in FIG. 6 can be kept separate from the injection unit 7 shown in FIG. 7 and assembled on the latter by the user just before use. In this configuration, a cap 8 of plastic such as polypropylene closes the vial 5 to keep the area of the plunger 51 clean, since it will touch the cannula 24. The user removes the cap 8 from the vial 6, pulls the injection unit 7 out of its protective package such as a polyvinyl chloride blister pack with a polyethylene peel film and screws its threads 38 the threading 51 of the plunger 4. The device is then ready to be activated and used.

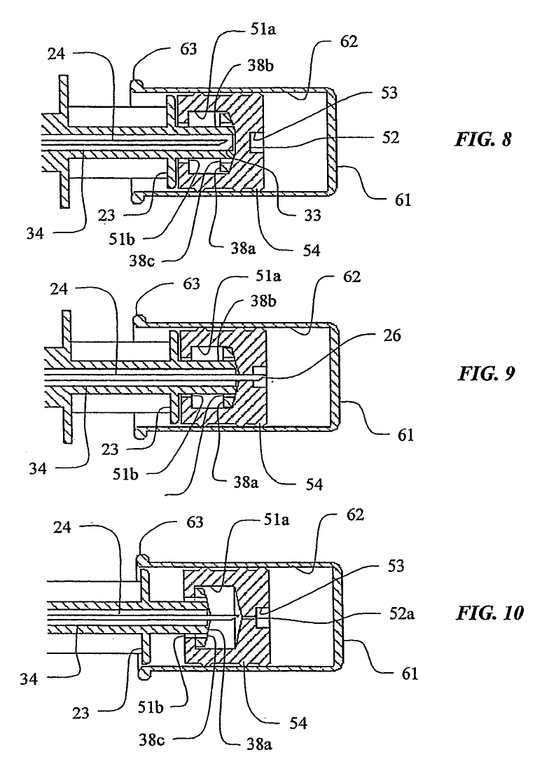

FIGS. 8, 9 and 10 show a second version of the preferred configuration in which the device cannot be reused. In this case, the plunger 4 has no female threading, but is provided with a rail 51a and a hole 51b with a smaller diameter than the rail 51a. A disk 38a located near the membrane 33 of the activation head 3 replaces the threading 38 and has a similar diameter to the rail 51a of the plunger 4 and can slide inside it. A cylinder 38b has a slightly smaller diameter than the hole 51b of the plunger 4. This disk 38a fits in the plunger 4, with the same type of assembly as is used to secure the pressure rods to the plungers in ordinary disposable plastic syringes. The disk 38a is run through by one or more grooves 38c. Looking at FIG. 9, when the activation is over, the inner point 26 of the cannula 24 comes out of the membrane 33 and pierces the membrane 52 of the plunger. During injection, the cannula 24 plunges into the liquid reservoir. But, when one tries to draw the liquid from outside, pushing the vial 5 away from the activation head 3 to create a vacuum between the vial 5 and the plunger 4, the ring 38a moves away from the membrane 52 of the plunger and the inner point 26 of the cannula 24 withdraws from it. Because of this, nothing can get into the vial 5 apart from the air flowing through the grooves 38c of the disk 38a. Therefore, the syringe cannot be reused. In other respects, the second version configuration is the same as the first one.

As shown above, the inner point 26 of the needle never comes in contact with the external environment. This is a very important advantage of this invention. Furthermore, an unintentional activation of the system due to accidental pressure on the needle-shield 1 would be immediately obvious through the window 45, since the hub 21 is no longer aligned with the level of the window 45. It is also possible to build a threaded activation system, the advantage being that such a system would not be activated by accidental pressure, such as a bump. However, a threaded system may be more difficult to use. On the other hand, the pressure activated system is easy to use and the integrity of the device is immediately verified by direct visual control.

Another advantage of this invention is that a level gauge on the injection unit, as shown on the flap 35 of the activation head 3, permits disposal of part of the liquid contents in order to inject a pre-defined fraction of a dose, such as a pediatric dose.

It should be noted that the activation indicator could also consist of two horizontal parallel dark lines, rather than the window 45. However, the window allows the user to have a better view of the position of the hub 21 in case the material of the activation head is not perfectly transparent.

Thus, it is apparent that there has been provided, in accordance with the invention, an activation-based injection device that fully satisfies the objects, aims and advantages set forth above. While the invention has been described in conjunction with a specific embodiment thereof, it is evident that many alternatives, modifications and variations will be apparent to those skilled in the art and in light of the foregoing description. Accordingly, it is intended to embrace all such alternatives, modifications and variations as fall within the spirit of the appended claims.

What is claimed is:

1. A device for injecting a drug into a patient comprising:
an activation head (3) open at one end thereof and closed by a pierceable membrane (33) at another end thereof;
a needle-shield (1) closed at one end (11) thereof and open at a base (13) thereof, said base (13) fitting and sliding in a rail (31) of said activation head (3);
a rigid vial (5) having a reservoir containing the drug and having a sliding plunger (4) hermetically sealing said vial, said plunger adapted for attachment to said another end of said activation head (3) for making direct contact between a pierceable membrane of said plunger (52) and said pierceable membrane of said activation head (33);
a needle (2) having a double-pointed cannula (24) secured to a hub (21) disposed inside said rail of said activation head (3);
an activation indicator (45) on said rail of said activation head (3) for indicating an initial resting position of said hub (21) of said cannula (24) in said activation head (3) in which said pierceable membrane (33) is intact; and
a needle-shield (1) slidable inside said activation head (3) from said initial resting position to a final activation position for acting on said hub (21) and sequentially piercing said pierceable membranes (33 and 52) with said inner point (26) of said needle (2) and connecting said cannula (24) to said reservoir of said vial (5).

2. A device according to claim 1, said activation head (3) being at least semitransparent and said activation indicator (45) being a window (45) in which said rail of said activation head is thinner so as to offer a better view of said needle hub (21) inside said rail.

3. A device according to claim 1, said rail of said activation head (3) containing two retainers (40 and 41) for stopping said needle hub (21) before activation and for locking said needle hub (21) in position after activation.

4. A device according to claim 3, said needle-shield (1) having a thicker ring (12) acting as a stop against a top of said activation head (3) during activation.

5. A device according to claim 3, said rail (31) of said activation head (3) having an inner narrower diameter defining a stop (37) for allowing said cannula (24) to pass while stopping said hub (21).

6. A device according to claim 1, said needle hub (21) being colored.

7. A device according to claim 1, said activation head (3) having a flange (36) to grip the device during operation thereof.

8. A device according to claim 1 further comprising at least one level gauge (44) on said activation head (3) making the device usable for administration of both full doses and fractions of doses of the drugs.

9. A device according to claim 1, said activation head (3) being stiffened at said base by at least one radial flap (35).

10. A device according to claim 1, said vial (5) sealed by the plunger (4) being separated from said injection unit and stopped by a cap (8) which must be removed from said vial before assembling said plunger (4) to said another end (38) of said activation head (3).

11. A device according to 1, said vial (5) being transparent, said needle hub (21) and said activation head (3) being plastic and said needle shield (1) being of rigid transparent plastic.

12. A device according to claim 1, said base of said activation head (3) and said plunger (4) having complementary threading (38 and 51).

13. A device according to claim 1, said base (38a) of said activation head (3) and said plunger (51a) being free to slide into each other without locking, whereby, upon an attempt to draw liquid from outside, said cannula inner point (26) is pulled out of said plunger membrane (52) so that said vial (5) cannot be refilled.

14. A device according to claim 13, said base (38a) of said activation head (3) having one or more grooves (38c) permitting air to flow more smoothly into said vial (5) through an orifice (52a) made in said membrane (52) by said inner point (26) of said needle (2) during activation to draw new liquid into said vial.

15. A device according to claim 1, said plunger (4) being made of rubber.

16. A device according to claim 1, said activation head (3) having a smooth internal surface facilitating sliding of said needle hub (21) by pressing upon said needle-shield (1).

17. A device according to claim 1, said rail of said activation head (3) and said needle hub (21) having complementary threading and said base (13) of said needle-shield (1) and said hub (21) having at least one set of complementary notches whereby rotation of said needle-shield (1) screws said hub (21) into said rail of said activation head (3).

18. A device for injecting a drug into a patient comprising:
an activation head (3) closed at one end by a pierceable membrane (33);
a needle-shield (1) open at a base (13) thereof, said base (13) fitting and sliding in a rail (31) of said activation head (3);
a rigid vial (5) having a sliding plunger (4) sealing said vial, said plunger adapted for attachment to said closed end of said activation head (3) for making direct contact between a pierceable membrane of said plunger (52) and said pierceable membrane of said activation head (33);
a needle (2) having a double-pointed cannula (24) secured to a hub (21) disposed inside said rail of said activation head (3); and
a needle-shield (1) slidable inside said activation head (3) from an initial resting position to a final activation position for acting on said hub (21) and sequentially piercing said pierceable membranes (33 and 52) with said inner point (26) of said needle (2) and connecting said cannula (24) to said vial (5).

19. A device according to claim 18 further comprising an activation indicator (45) on said rail of said activation head (3) for indicating said initial resting position of said hub (21) of said cannula (24) in said activation head (3) in which said pierceable membrane (33) is intact.
